(19) United States
(12) Patent Application Publication
(10) Pub. No.: US 2020/0048319 A1
(43) Pub. Date: Feb. 13, 2020

(54) Compositions of GLIPR Fusion Proteins and Methods for the Treatment of Prostate Cancer

(71) Applicant: Glipper Oncology Research, Inc., Houston, TX (US)

(72) Inventors: Louis Eugene Burton, San Mateo, CA (US); Amita Saxena Goel, Pleasanton, CA (US); Divya Goel, Pleasanton, CA (US)

(73) Assignee: Glipper Oncology Research, Inc., Houston, TX (US)

(21) Appl. No.: 16/534,285

(22) Filed: Aug. 7, 2019

Related U.S. Application Data
(60) Provisional application No. 62/715,465, filed on Aug. 7, 2018.

(51) Int. Cl. C07K 14/47; A61K 47/68; A61P 35/00

(52) U.S. Cl. CPC C07K 14/4748; A61K 38/00; A61P 35/00; A61K 47/68

(57) ABSTRACT

This invention is directed to fusion proteins comprised of one or more protein sequences of the glioma pathogenesis-related (GLIPR) family of proteins coupled to non-GLIPR protein sequences, to nucleic acid constructs and vectors comprising encoding fusion proteins and peptides, and to methods related to fusion proteins and peptides in the treatment of diseases. In particular, the invention is directed to GLIPR sequences coupled to sequences of antibodies and/or other immune system proteins and peptides, and methods related to fusion proteins in the treatment of prostate cancer.

### REFERENCE TO RELATED APPLICATIONS

This application claims priority to U.S. Provisional Application No. 62/715,465 entitled “Compositions of GLIPR Fusion Proteins and Methods for the Treatment of Prostate Cancer,” filed Aug. 7, 2018, the entirety of which is hereby incorporated by reference.

### BACKGROUND OF THE INVENTION

### 1. Field of the Invention

This invention is directed to fusion proteins comprised of one or more protein sequences of the glioma pathogenesis-related (GLIPR) family of proteins coupled to non-GLIPR protein sequences, to nucleic acid constructs and vectors comprising encoding fusion proteins and peptides, and to methods related to fusion proteins and peptides in the treatment of diseases. In particular, the invention is directed to GLIPR sequences coupled to sequences of antibodies and/or other immune system proteins and peptides, and methods related to fusion proteins in the treatment of prostate cancer.

### 2. Description of the Background

Prostate cancer is characterized as the growth of prostate epithelial cells to form one or more tumors and is a leading cause of cancer deaths among males in industrialized countries. The incidence and mortality of prostate cancer increase with age with surprising differences between racial groups. Prostate cancer exists is two general forms, benign prostate hyperplasia (BPH) and metastatic cancer. BPH is not considered malignant, but a growth of untransformed cells that remain confined to the prostate. Prostate cancer that metastasizes typically attacks the bone and/or lungs, but can form tumors most anywhere in the body.

Surgery and/or radiotherapy remain the treatments of choice for early stage prostate cancer. Surgery involves complete removal of the prostate (prostatectomy) typically with extractions of surrounding lymph nodes. Radiotherapy involves treatment of the affected tissue by exposure to radioisotopes. As prostate cells require hormone exposure, endocrine therapy is often a treatment of choice for early or late stage cancers, which involves depriving prostate cells of testosterone. This is often performed by anti-androgen therapy; administering antiandrogens (e.g., flutamide and bicalutamide), estrogens, or synthetic hormones that are agonists of luteinizing hormone-releasing hormone. These molecules inhibit testicular and organ synthesis and suppress luteinizing hormone secretion which in turn leads to reduced testosterone secretion by the testes.

These therapies are palliative and have numerous side effects. Second generation androgen signaling inhibitors, abiraterone, and enzalutamide, have been shown in clinical trials to extend survival, but these therapies are not curative and largely only delay the metastatic spread of cancerous cells. Following the diminished effects of anti-androgen therapy, chemotherapy (e.g., docetaxel) provides only modest survival benefits where prostate cancers continue to grow. Although immunotherapy has shown dramatic responses in melanoma and subsets of lung and urothelial cancers, thus far results for immunotherapy of prostate cancer have been refractory.

In its more aggressive form, transformed prostatic tissues escape from the prostate capsule and metastasize invading locally and throughout the bloodstream and lymphatic system. Metastasis, defined as tumor implants which are discontinuous with the primary tumor, can occur through direct seeding, lymphatic spread, and hematogenous spread. All three routes have been found to occur with prostate cancer. Local invasions typically involve the seminal vesicles, the base of the urinary bladder, and the urethra. Direct seeding occurs when a malignant neoplasm penetrates a natural open field such as the peritoneal, pleural or pericardial cavities. Cells seed along the surfaces of various organs and tissues within the cavity or can simply fill the cavity spaces. Hematogenous spread is typical of sarcomas and carcinomas. Hematogenous spread of prostatic carcinoma occurs primarily to the bones, but can include massive visceral invasion as well. A number of newly diagnosed prostate cancer patients will have metastases at the time of initial diagnosis.

When prostate cancer spreads beyond the confines of the prostate gland, transformed prostate cells metastasize to distant sites, predominantly bone and ling tissue, the disease is very difficult to control. In the spread of prostate cells throughout the body, the cells acquire specific changes in their genes. The changes that occur in the genetic make-up of prostate cancer cells allow these cells to grow and to spread throughout the body, and specifically to be able to survive and expand within the bone microenvironment.

p53 is a tumor suppressor gene whose mutation is commonly associated with the transformation of cells including prostate cells. Mutation in p53 gene can lead to cellular malfunctions such as malignant growth and metastasis or cells including prostate cells. Numerous studies have demonstrated a correlation between loss of p53 function and metastasis of prostate cancer cells.

In a mouse model, loss of p53 function led to the development of metastases that seed from cells within the prostate tumor. This suggests that p53 mutations may allow for metastases that clonally expand to distant sites. Additional studies demonstrated that specific p53 mutations are clonally expanded in metastatic prostate cancer, and that a pattern of aberrant p53 expression in primary tumors, termed clustered p53 staining, has significant prognostic value in predicting recurrence following radical prostatectomy. It is generally considered that the nature of functional alterations which occur in cells containing p53 mutations specifically facilitates metastatic seeding, survival, and growth at distant metastatic sites. These alterations likely result, in part, from aberrant regulation of genes under the transcriptional control of p53 that have previously been shown to mediate apoptosis and anti-angiogenic activities.

A p53 target gene with tumor suppressor functionality was identified and referred to the glioma pathogenesis-related (GLIPR) family of proteins found in both mice (e.g., GLIPR1, GLIPR1/1, GLIPR1/2, GLIPR1/3,) and humans (e.g., GLIPR1, GLIPR1L1, GLIPR1L2, GLIPR1alpha, GLIPR1 beta, GLIPR2alpha, GLIPR2beta, GLIPR2gamma, GLIPR2delta, GLIPR2episilon). GLIPR is a member of the pathogenesis-related protein (PR) superfamily which includes the proteins related to testes-specific, vespid, and pathogenesis protein 1 (RTVP1), and the cysteine-rich secretory protein (CRISP) family (Chengzhen Ren et al., Identification and characterization of RTVP1/GLIPR1-like genes, a novel p53 target gene cluster. Genomics 88:163-172, 2006, which is specifically and entirely incorporated by reference).

In contrast to normal prostate tissue, the human GLIPR1 promoter was found to be highly methylated in prostate cancer tissue. High degrees of methylation correlated with the decreased level of GLIPR1 expression and uncontrolled cell proliferation. Consequently, GLIPR1 was proposed to act as a tumor suppressor that undergoes epigenetic inactivation in prostate cancer and as a possible target in the treatment of prostate cancer. Additional studies showed that GLIPR1 expression is induced by DNA-damaging agents independent of p53.

The GLIPR1 protein contains a SCP-like (single cell protein-like) extracellular domain and a structurally conserved, cysteine-rich secretory domain (CAP). The GLIPR1 protein exists in two isomeric forms identified as P48060-1 and P48060-2, which, respectively, encode 266 amino acids (30.366 kDa) and 236 amino acids (26.919 kDa). GLIPR1 is highly tissue specific with high levels of mRNA expression in testes with little to no expression in normal cells of the bladder, prostate, kidney, lung, and bone marrow.

Preclinical studies showed a significant suppression of tumor growth when GLIPR1 protein was directly injected into prostate cancer cells using an immunocompetent orthotopic mouse model. Functional analysis of GLIPR1 revealed both growth suppression and pro-apoptotic activities for both mouse and human GLIPR1 in multiple different cancer cell lines. The pro-apoptotic activities showed sustained c-Jun-NH(2) kinase signaling. Transduction of an adenoviral vector-mediated GLIPR1 protein (AdGlipr1) into prostate cancer tissues using an immunocompetent orthotopic mouse model showed biologic activities that correlated with tumor-suppressor functioning as well as a significant reduction of tumor-associated angiogenesis and an apparent direct suppression of endothelial-cell sprouting activities. In addition, AdGlipr1 strongly stimulated antitumor immune responses and produced in cytotoxic T-lymphocyte activation. In addition, administration of a tumor cell vaccine of GLIPR protein 1 showed antitumor activity in a mouse model of recurrent prostate cancer.

These data directly indicate that expression of GLIPR protein can play a significant role in prostate and other forms of cancer. A number of U.S. patents have issued directed to GLIPR1 proteins and method of cancer treatment (all of which are specifically and entirely incorporated by reference): U.S. Pat. No. 7,601,806 is directed to sequences of GLIPR1 proteins and nucleic acid that encode GLIPR proteins for administration to patients with prostate cancer. U.S. Pat. No. 7,645,452 is directed to compositions and methods which involve GLIPR overexpression. Experiments are disclosed using both mouse or human cells showing that administration of GLIPR1 leads to apoptosis and the down regulation of prostate cancer. U.S. Pat. No. 7,723,475 is directed to purified and isolated GLPR1 peptides and nucleic acids encoding such polypeptides. U.S. Pat. No. 7,824,685 is directed to comparative differential display and co-transfection technologies using GLIPR1 mRNA in normal mouse and human cells verses corresponding metastatic cells. These studies indicate a direct role for GLIPR1 protein is metastatic cell stimulated apoptosis. The anti-angiogenic therapeutic effects observed were associated with an increased local and systemic immune response against prostate tumor cells. Although various GLIPR proteins have been produced and used recombinantly, obtaining long term expression sufficient to provide long-term therapeutic benefit has been elusive. The half-life of GLIPR1-TM is believed to be too short for a therapeutic effect (typically 60 minutes or less) or a lasting therapeutic effect which results in an increased frequency of dosing and/or increased dosing quantities to be effective, if at all.

Accordingly, there is a need for improved research tools, diagnostic tools, and therapies, useful for the diagnosis, treatment and prevention of prostate cancer, bladder cancer, various metastatic diseases, and other maladies.

### SUMMARY OF THE INVENTION

The present invention overcomes the problems and disadvantages associated with current strategies and designs and provides new proteins and related products, and methods for preventing and/or treating diseases such as prostate cancer.

One embodiment of the invention is directed to recombinant peptides comprising a first amino acid sequence of a GLIPR protein coupled to a second amino acid sequence unrelated to the GLIPR protein. Preferably the first amino acid sequence comprises a conserved region of the GLIPR protein, which is preferably of human or murine origin, wherein the first amino acid sequence comprises a domain of the GLIPR protein. Preferably the domain comprises a trans membrane or secretory domain. Preferably the human GLIPR protein comprises GLIPR1, GLIPR1L1, GLIPR1L2, GLIPR1 alpha, GLIPR1 beta, GLIPR2alpha, GLIPR2beta, GLIPR2gamma, GLIPR2delta, or GLIPR2episilon. Preferably the second amino acid sequence comprises a sequence obtained or derived from an antibody, an immunological protein, an immune-regulatory protein, a cytokine, or a toxin protein. Preferably the antibody is an IgA, an IgD, an IgE, an IgG, or an IgM, and also preferably, the second amino acid sequence is obtained or derived from an Fc region of the antibody. Preferably the peptide comprises the amino acid sequence of any one of SEQ ID NOs 1-17. Preferably the peptide has a circulating half-life of at ten times the circulating half-life of the GLIPR protein. More preferably at least one hundred time the circulating half-life of the GLIPR protein.

Another embodiment of the invention is directed to first amino acid sequence of a GLIPR protein coupled to at least two second amino acid sequences unrelated to the GLIPR protein. The first non-GLIPR sequences serves to stabilize the compound and the second provides an added functionality. Functionalities that would be useful in association with GLIPR treatments include stimulation of the immune system such as macrophage and/or T cell activation.

Another embodiment of the invention is directed to recombinant nucleic acids that encodes one or more peptides of the invention. The recombinant nucleic acid may be an expression vector that preferably contains the sequences for expression of the peptide such as, for example, transcription and translation initiation sequences, enhancer sequences, induction sequences, splicing sites, and additional sequences needed or desired for maximal expression of the peptide.

Another embodiment of the invention is directed to methods for treating a patient comprising: providing a composition containing the peptide of claim 1; and administering a therapeutic amount of the composition to the patient. Preferably the patient has a disorder associated with an uncontrolled growth of cells such as, for example, a malignancy or a tumor. Preferably the malignancy or tumor comprises bone, bladder, kidney, liver, lung, or prostate cancer. Preferably the patient has a reduced expression of GLIPR1 protein in cells as compared to similar cells in which GLIPR1 protein expression is normally enhanced. Preferably administering comprises systemic administration to the bloodstream of the patient or local administration.

Another embodiment of the invention comprises peptides containing a first amino acid sequence of a GLIPR core protein coupled to a second amino acid sequence comprising a Fc portion of an antibody. Preferably the peptide has a circulating half-life that is significantly greater than the circulating half-life of the GLIPR core protein, more preferably at least ten times greater, and more preferably at least one hundred times greater than the circulating half-life of the GLIPR core protein. Also preferably, the peptide comprises the sequence of any one of SEQ ID NO. 1-17.

Other embodiments and advantages of the invention are set forth in part in the description, which follows, and in part, may be obvious from this description, or may be learned from the practice of the invention.

### DESCRIPTION OF THE FIGURES

FIG. 1 Human GLIPR-del TM (with myc and His tag) in psecB.

FIG. 2 Four variations of Core GLIPR1 Protein.

FIG. 3 Four variations of the Fc Fusion protein.

FIG. 4A The amino acid sequences of Fc-Glipr1 with +DAAP/+RNR, with +DAAP/−RNR, with −DAAP/+RNR, and with −DAAP/−RNR.

FIG. 4B The amino acids sequence of Fc-GLIPR1 with glycine linker, without linker, with His-Tag GLIPR, and native GLIPR.

FIG. 5A Schematic showing the GLIPR1 protein receptor.

FIG. 5B Schematic showing an IgG1 protein.

FIG. 5C Schematic showing a fusion of the Fc portion of an IgG1 antibody and the GLIPR1 protein receptor.

FIG. 6A Schematic showing the removal of Glipr1's transmembrane domain (GLIPR1 delta TM).

FIG. 6B Schematic showing elimination of the antibody Fab fragment.

FIG. 6C Schematic showing the Fc portion of the antibody fused to the C-terminus of GLIPR1delta TM with dimerization of GLIPR1-Fc fusion.

FIG. 7 Cleavage of antibody with pepsin or papain.

FIG. 8 Generation of F(ab)2 version of Fc-GLIPR1.

### DESCRIPTION OF THE INVENTION

The expression of GLIPR1 protein in human prostate cancer and metastatic prostate tumors is significantly reduced relative to expression in normal prostate tissues. GLIPR1 protein is believed to be a tumor suppressor protein that acts as a master switch to suppress multiple molecular pathways that drive prostate cancer. GLIPR1 expression induces apoptosis of cancer cells directly, and also modifies the microenvironment that surrounds prostate cancer cells to promote prostate cancer destruction. This is believed due, in part, to an increase in cell cancer destruction and a decrease of angiogenesis, the ability to generate blood vessels and immune cells to feed the tumor. GLIPR1 protein also promotes infiltration of CD8 and CD4 T cells into prostate cancer tissues and induces an anti-tumor immune response. In addition, the direct and indirect effects of adenoviral vector-mediated GLIPR1 gene therapy treatment are seen on direct intra-prostatic injection in a clinical trial. The results achieved demonstrate a direct cytopathic effect, and induction of Th1 immune responses at the injection site and also systemically.

When administered to a patient, the GLIPR1 protein has a relatively short half-life, surviving intact in the bloodstream for a short time, typically only minutes. A half-life of minutes is typically too short for a therapeutic effect, or a local or systemic effect, and/or any sort of long-term therapeutic effect. To address the short half-life, administration can be more frequent and/or quantities per dose can be increased. For GLIPR1, the frequency and quantities cannot be raised sufficiently to achieve the desired therapeutic effect, and also, unwanted side effects negate or overwhelm the positive therapeutic effect of the administration.

It was surprisingly discovered that forms of GLIPR1 protein coupled to non-GLIPR sequences stabilize the GLIPR protein and increase the half-life and effectiveness of GLIPR without compromising the therapeutic result to be achieved. The resulting compound also induces apoptosis of cancer cells, is selectively taken up by cancer cells, and has associated anti-angiogenic effects including a local and systemic immune response in the form of an anti-tumor effect against the cancer. Coupling of GLIPR to non-GLIPR amino acid sequences can occur by recombinant engineering techniques using nucleic acid constructs that encode the desired sequences, by enzyme action, or by chemical coupling such as conjugation. Adding different portions of molecules together can increase size, impart characteristics of one molecule, the other molecule, or both molecules to the final construct, but the result is not known until testing as there is no way to predict an outcome. Thus, it was surprisingly determined that the C-terminal His tagged GLIPR1 had both activity and stability, and did not interfere with biological function. Recombinant engineering involves creating an expression vector containing the GLIPR and non-GLIPR nucleic acid sequences aligned in a vector for proper expression in a prokaryotic (e.g., E. coli) or eukaryotic (e.g., yeast, mammalian) expression system. Preferably, the expression vector contains a ribosome binding site, transcription and translation initiation sites, and an ATG codon, and optionally an enhancer sequence that upregulates expression and/or an inducer that allows for expression when the cells are exposed to an inducing agent. Examples of recombinant engineering techniques are disclosed and described in, for example, U.S. Pat. Nos. 7,348,408; 8,975,041; 9,738,699; 10,000,550 (the disclosures of which are each incorporated by reference). Enzyme action involves subjecting the GLIPR and non-GLIPR sequences to an appropriate enzyme that couples the sequences (e.g., ligases) such as is disclosed and described in, for example, U.S. Pat. Nos. 6,132,722 and 7,361,487 (the disclosures of which are each incorporated by reference). Conjugation refers to chemical coupling. Typical coupling agents include, for example, carbodiimide, amino oxy reagents (e.g., oxime chemistry), cyano agents (e.g., 1-cyano-4-dimethylaminopyridine tetrafluoroborate, 2-cyanopyridazine-3(2H)one, 1-cyanobenzotriazole, 1-cyanoimidazole, 1-cyano-4-pyrrolidinopyridinium tetrafluorborate), and may include linkers, tags (e.g., HIS tags, CYS tags), and chemical modifications as necessary to allow for coupling. Chemical coupling is well known in the art with many examples disclosed and described in, for example, U.S. Pat. Nos. 5,651,971; 5,693,326; 5,849,301; and 9,044,517 (the disclosures of which are each incorporated by reference). Coupled GLIPR/non-GLIPR amino acid sequences of the invention are referred to herein as fusion proteins regardless of the method of construction.

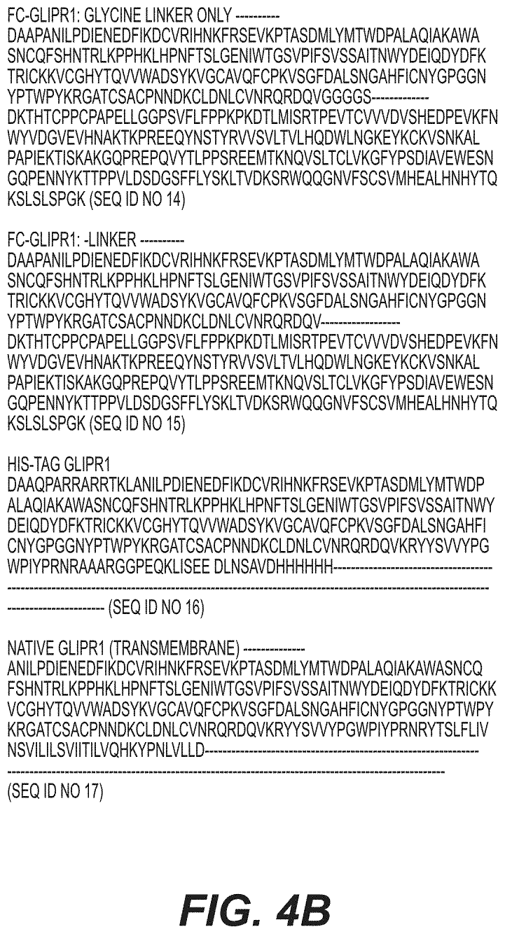

Coupling is preferably covalent, for example, through amino to carboxy linkages or linker molecules or through conjugation. Linker molecules include, for example, 0 and N linked glycan linkers. Alternatively, coupling may be ionic (e.g., hydrogen bonding), stearic, or van der Waals forces. The GLIPR sequences that can be coupled include all or the functionally active portion of a GLIPR protein, which includes but is not limited to the core sequence, the trans-membrane domain, the secretory domain, the extracellular domain, or a functional subdomain thereof, and combinations thereof. Preferably included are any sequences that are at least 50% identical, at least 60% identical, at least 70% identical, at least 80% identical, at least 90% identical, at least 95% identical, or at least 99% identical to a GLIPR sequences (e.g., preferably human), and any sequences that include conserved or non-interfering mutations or functional regions of the GLIPR sequence. Non-GLIPR sequences that can serve as fusion partners to GLIPR include, for example, all or portions or peptides or proteins that are known to be stable and/or have significantly longer circulating half-lives as compared to the GLIPR proteins. Examples of stable non-GLIPR amino acid sequences include sequences of all or a portion of a protein or peptide of the immune system such as an antibody sequence or a portion thereof, an immune-regulatory protein sequence, and/or a cytokine sequence, a toxin protein, or combinations thereof. Preferably included are any sequences that are at least 70% identical, at least 80% identical, at least 90% identical, at least 95% identical, or at least 99% identical to a non-GLIPR sequences, and any sequences that include conserved or non-interfering mutations or functional subdomains thereof. Preferred examples include antibody proteins such as sequences of an IgA, IgD, IgE, IgG or IgM. Also preferred are sequences of immune-regulatory proteins such as interleukins, cytokines, interferon, and combinations thereof. Certain toxin proteins increase stability and have the additional benefit of being capable of stimulating and targeting the immune system and can also be detoxified without compromising their effects on the immune system. Examples of such toxin proteins include diphtheria toxoid (e.g., CRM₁₉₇), tetanus toxoid (e.g., tetanus toxoid heavy chain fragments, TTHc) and combinations thereof. A preferred fusion protein of the invention comprises but is not limited to the GLIPR 1 core sequence (GLIPR1-ΔTM) covalently coupled to an antibody Fc sequence of IgG1 (see FIGS. 5A, 5B, and 5C).

Preferred GLIPR fusion proteins also include, but are not limited to fusion proteins comprising core GLIPR1 protein sequences coupled to non-GLIPR sequences such as sequences of antibody molecules. Preferred fusion proteins of the invention may also contain more than a single non-GLIPR amino acid sequence. For example, one non-GLIPR sequence may be included to provide stability and another non-GLIPR sequence included to provide a further functionality (e.g., metal binding or activated) and improved expression (e.g., the sequence DAAP coupled to the 5′terminus). Further functionalities include, but are not limited to activation of the immune system such as recruiting phagocytes or natural killer cells to a specific site. Also, an additional toxoid can induce T cell activity.

GLIPR fusion proteins may also be modified by posttranslational and/or post-isolation enzymatic modifications of the expressed fusion form of the molecule. By modification of the splicing signals, altered forms of GLIPR may be created. By enzymatic treatment, the fusion protein may be modified with, for example, amino groups, methyl groups, carboxy groups, reducing agents, oxygenizing agents, pH modifications, linkers, coupling agents for selective binding (e.g., biotin, avidin, streptavidin), labeling agents for detection, agents that promote the manufacture and/or isolation of the fusion construct itself (e.g., protein A, biotin, avidin, streptavidin), cell killing agents (e.g., anti-tumor agents, anti-metastatic agents, immunological molecules), agents that promote an immunological response, and/or probes that can themselves be detected. These modifications can increase stability, allow for monitoring or create positive functional attributes to the construct not otherwise available absent the modification.

Stability of GLIPR and GLIPR fusion products can be easily determined from circulating half-life of the compound after injection. Preferably, stability and/or circulating half-life of the fusion protein increased the circulating half-life of the molecule from minutes without its fusion partner, to hours when coupled. Preferably, stability of the fusion product was at least one hour, preferably 2 hours or more, 3 hours or more, 4 hours or more, 5 hours or more, 6 hours or more, 12 hours or more, 18 hours or more, 24 hours or more, days or more, weeks or more, or a month or longer. Preferably the circulating half-life of the GLIPR fusion product increases the half-life of the original GLIPR protein 10 fold or more, 20 fold or more, 50 fold or more, 100 fold or more, 250 fold or more, 500 fold or more, 1,000 fold or more, or greater.

Fusion proteins identified and characterized can also be manufactured recombinantly or synthetically. Synthetic manufacture preferably comprises chemically coupling or amino acids to form the desired sequence. Recombinant manufacture preferably comprises creation of a nucleic acid vector that contains the desired portion of the GLIPR protein and a nucleic acid vector containing the desired sequence of the fusion partner, and utilizing restriction enzymes, and possibly tags and/or linkers, covalently coupling the GLIPR sequence with the non-GIPR sequence into an expression vector suitable for expression in an expression system. Expression systems are well-known and commercially available in a wide variety of cell types including bacteria, such as an E. coli expression system, a yeast expression system, and other prokaryotic or eukaryotic expression systems. Preferably the peptides of the invention are manufactured under good manufacturing practices (GMP) approved by the U.S. Food and Drug Administration, the European Medicines Agency (EMA), the Department of Health Canada, or appropriate local authority. Preferably, the recombinant nucleic acid encodes DAAP at the N-terminal sequences, which increases expression levels.

Another embodiment of the invention comprises administration of fusion proteins of the invention to a patient. Although the fusion proteins may be maintained in a dry (e.g., lyophilized) or liquid (aqueous or non-aqueous) state until use, preferably the fusion proteins are administered as a liquid composition which may include one or more pharmaceutically acceptable carriers. Administration may be systemic, such as via I.V., IM, SQ/SC, or I.P. injection or infusion, or localized to a specific site or cell structure of the patient such as a tumor. Preferably the effective amount is administered to create the desired therapeutic affect which may be, for example, suppression or eradication of a malignancy or a tumor.

Diseases and disorders that can be treated with compositions of the invention include, for example, malignancies, metastatic diseases, tumors, and any uncontrolled cell growth. Preferred diseases include, but are not limited to bone, bladder, kidney, liver, lung, brain and prostate cancer. Metastatic cancers may be found in virtually all tissues of a patient in late stages of the disease. For example, metastatic prostate cancer is found in seminal vesicles, lymph system/nodes (lymphoma), in bones, in bladder tissue, in kidney tissue, liver tissue and in virtually any tissue, including brain (brain cancer/tumor). Prostate cancers that can be treated also include BPH (benign prostatic hyperplasia) and hormone refractory prostate cancer. Compositions of the invention are generally applicable and may be used to treat any cancer in any tissue, regardless of etiology.

In general, the invention disclosed herein is directed to compositions and methods related to GLIPR fusion proteins including nucleic acids, polypeptides, and antibodies, for use in the treatment, prevention and detection of neoplastic disease and, specifically, metastatic prostatic neoplasia. The fusion proteins show little to toxicity when administered at therapeutic doses systematically in various animal cancer models. Results achieved with various animal models showed significant tumor suppression or outright eradication of the tumor. Preferably tumor suppression is at least 50%, preferably at least 60%, preferably at least 70%, preferably at least 80%, preferably at least 90%, preferably at least 99% and more preferably eradication.

Another embodiment of the disclosure is directed to antibody-drug conjugates or ADCs combining a GLIPR protein or smaller GLIPR peptide the targeting ability of an antibody, preferably a monoclonal antibody. ADCs are a class of biopharmaceutical drugs designed as a targeted therapy for treating cancer. Unlike chemotherapy, ADCs are intended to target and kill tumor cells while sparing healthy cells.

Another embodiment of the disclosure is directed to antibodies to all or a portion of the GLIPR protein such as disclosed herein. Preferably the antibodies are monoclonal antibodies and the disclosure include hybridoma cell lines that express the monoclonal antibody. Antibodies to GLIPR are preferably mouse-derived, human-derived or humanized antibodies.

The following examples illustrate embodiments of the invention but should not be viewed as limiting the scope of the invention.

### EXAMPLES

### Example 1

### Preparation of GLIPR1-deltaTM-Fc Fusion Protein

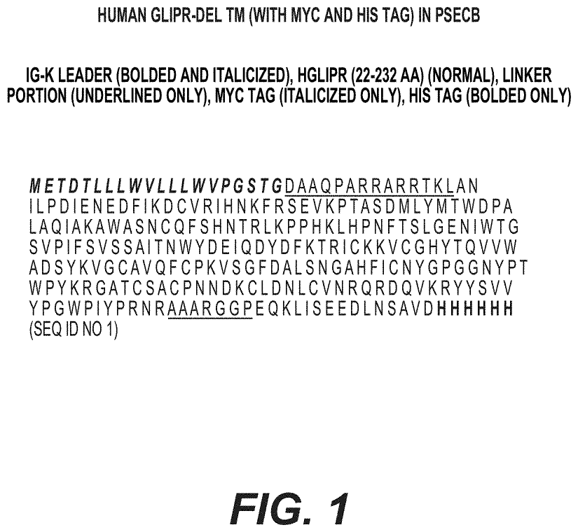

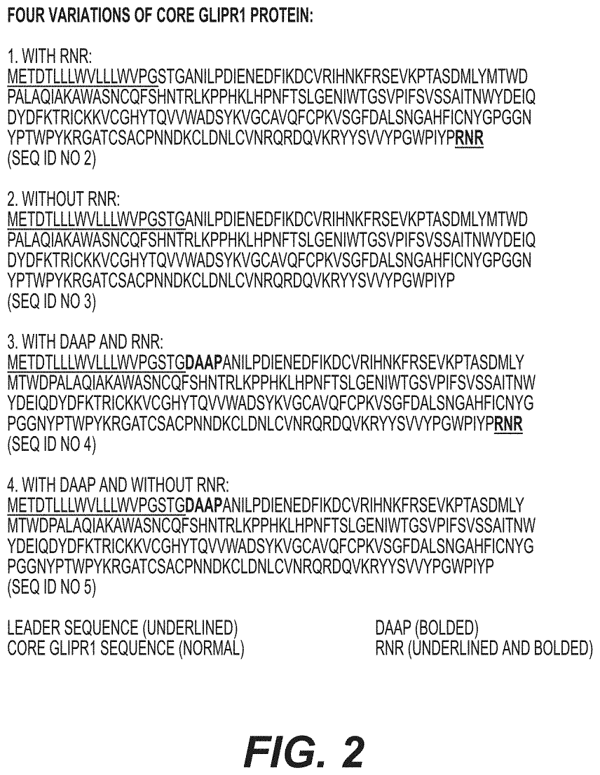

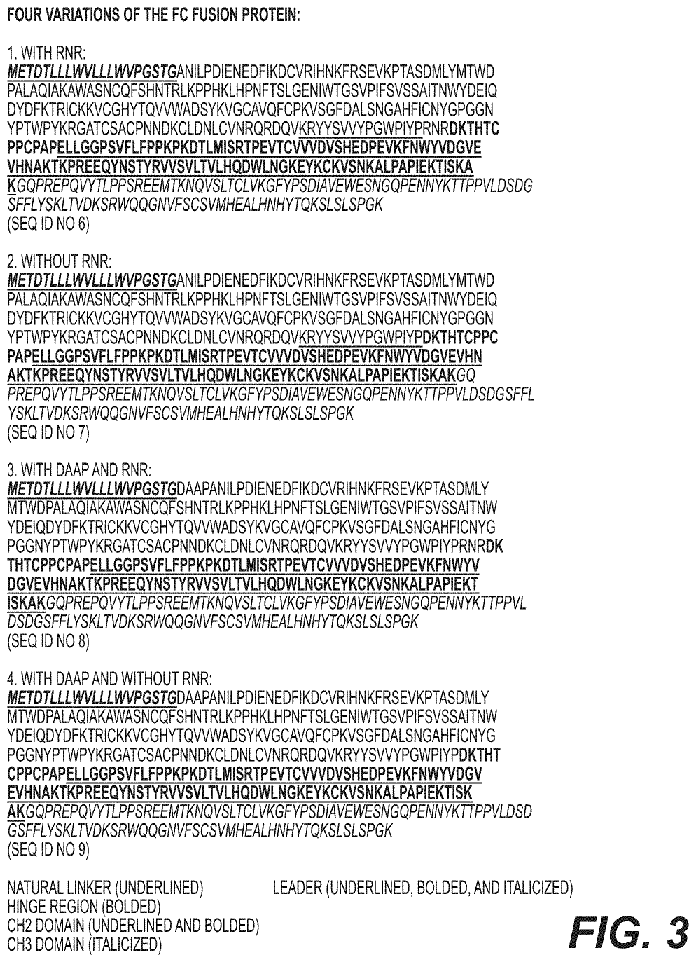

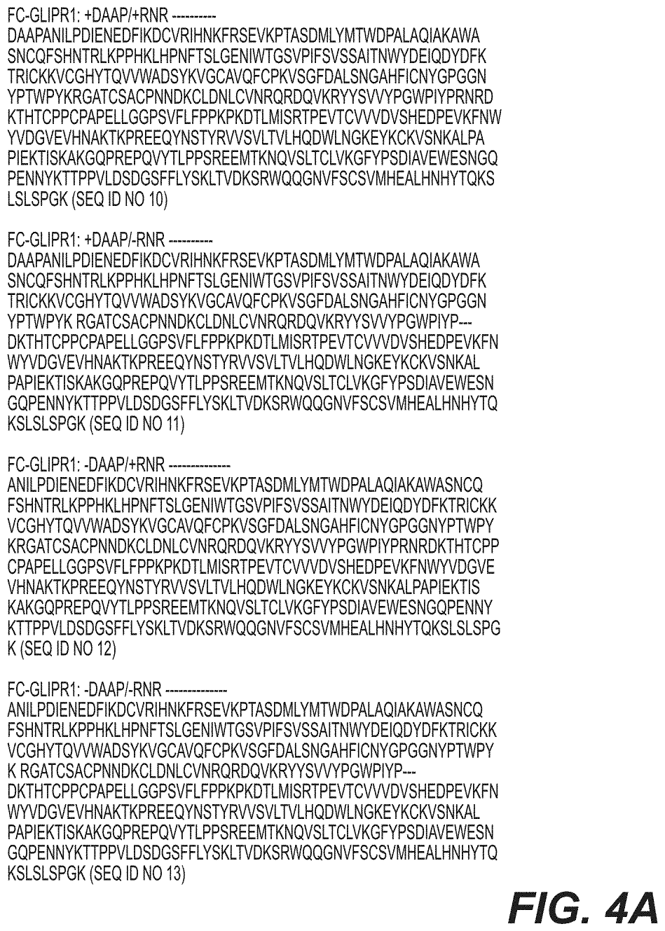

Experiments were performed to couple a sequence to GLIPR1-TM to increase the GLIPR1-TM molecule's half-life. Antibodies are natural products in the body having significant presence in the bloodstream. However, antibodies are large molecules comprised of a constant region (Fc region), a variable region (Fv), and a hypervariable region (Fhv). In a first experiment, only the Fc region of an IgG molecule was recombinantly coupled to GLIPR1-TM. Various GLIPR fusion proteins were created. (see FIGS. 1-4). The presence of the Fc portion also enabled the use of affinity purification (Protein A) of the fusion proteins to a high degree.

### Example 2

### Fusion Protein Characterization

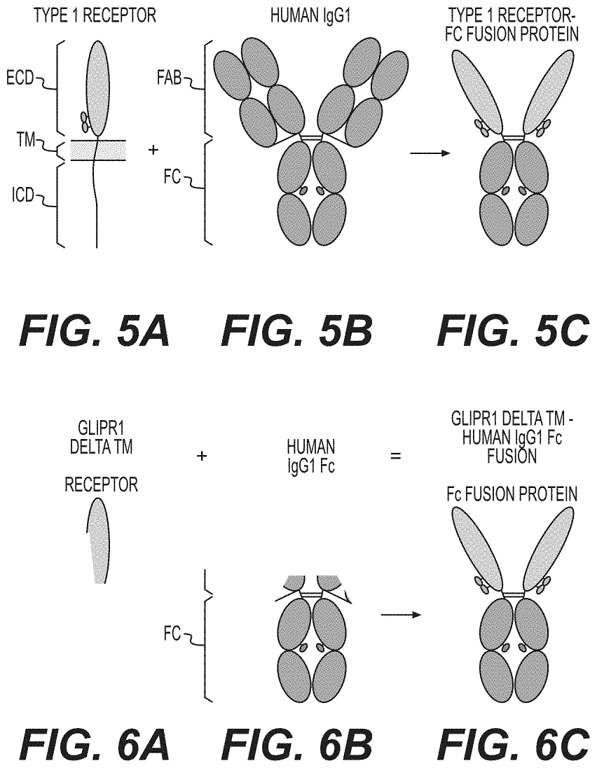

The fusion proteins created in Example 1 were characterized to determine the structures of the fusion products created. Schematics of the molecules created are shown in FIGS. 5A, 5B, 5C, 6A, 6B and 6C.

### Example 3

### Generation of F(ab)2 version of Fc-GLIPR1

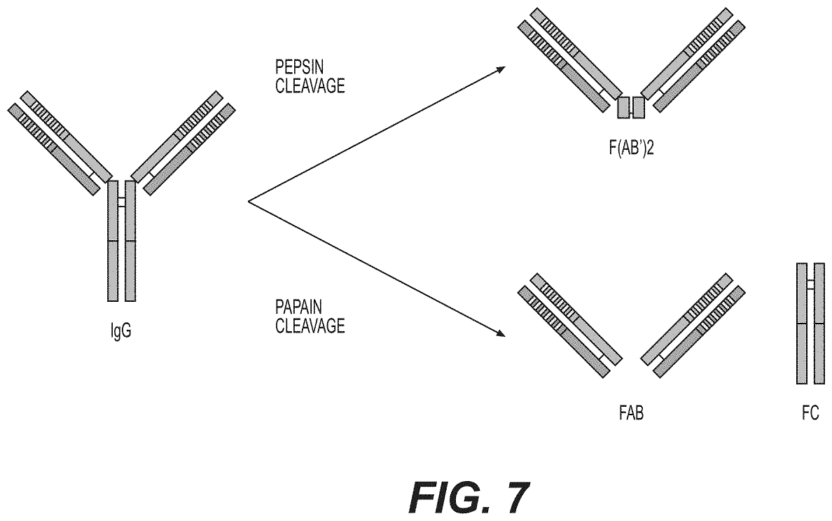

GLIPR1 protein produced recombinantly is digested with pepsin or papain to generate a dimeric GLIPR1 protein without the Fc region. Pepsin is a non-specific endopeptidase that is active at acid pH levels, and is irreversibly denatured at alkaline pH levels. Pepsin cleavage of the dimeric form of the fusion Fab′2 molecule generates a “dimeric” form of GLIPR1 connected by the residual hinge regions from each fusion monomer joined by a disulfide bridge (FIG. 7). Papain cleavage of the dimeric form of the fusion generates in a “monomeric” form of GLIPR1 with a C-terminal tail comprising the residual hinge region from each fusion monomer but not joined by a disulfide bridge (FIG. 7).

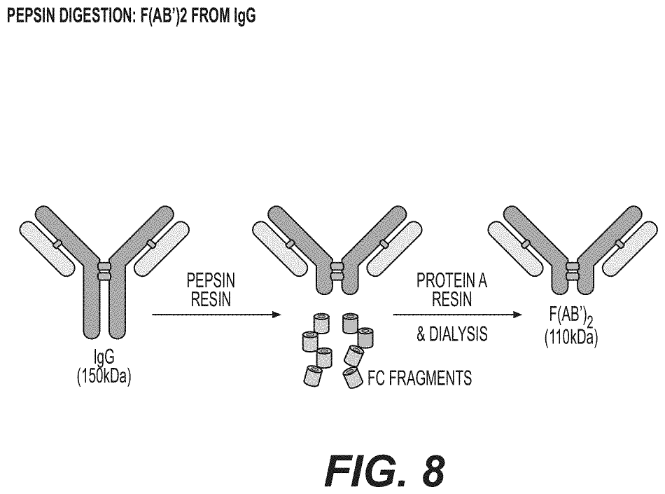

Digestion produces one F(ab)2 fraction and numerous small peptide of the Fc portion. The resulting F(ab)2 fragment is composed of two disulfide connected Fab units (FIG. 8). The Fc fragments is extensively degraded and those fragments are separated from the F(ab)2 fraction by dialysis, filtration or exchange chromatography

The F(ab)2 units can be separated by mild reduction (e.g., with 2-mercaptoethanamine-HCL or another reducing agent) into two sulfhydryl-containing univalent Fab fragments. An advantage of Fab fragments is that they can be conjugated to detectable labels directly through their sulfhydryl groups, ensuring that the active binding site remains unhindered and active. Alternatively, the sulfhydryl groups can be can be blocked with an alkylating reagent such as, for example N-ethylmaleimide (NEM) to prevent reformation of the F(ab)2 molecule.

### Example 4

### Treatment of Patients

The C-terminal his tagged human GLIPR1 fusion construct is expressed and isolated and purified by chromatography, and completely dissolved with a pharmaceutically acceptable oil at approximately 10.0 μg protein per mL. The formulation is administered subcutaneous or i.v., or via intramuscular injection (0.2-1.0 mL) to patients with metastatic prostate cancer stage II or higher. Dosing continues once a day for a period of 6-12 months. Tumor size is determined by MRI weekly. Reduction and/or elimination of tumors is observed in a majority of patients. PSA blood levels are measured weekly and decrease following the first month of treatment, initially by ten percent, and subsequently by ten percent per week thereafter leveling off at PSA values of 10.0 (ng/mL) or less in all patients treated.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims. Furthermore, the term “comprising of” includes the terms “consisting of” and “consisting essentially of.”

Sequence Information:

SEQ ID No 1: Human GLIPR-del TM(with myc and His
tag)in psecB [symbol] [symbol] [symbol]
DAAQPARRARRTKLANILPDIENEDFIK
DCVRIHNKFRSEVKPTASDMLYMTWDPALAQIAKAWASNCQFSHNTRL
KPPHKLHPNFTSLGENIWTGSVPIFSVSSAITNWYDEIQDYDFKTRIC
KKVCGHYTQVVWADSYKVGCAVQFCPKVSGFDALSNGAHFICNYGPGG
NYPTWPYKRGATCSACPNNDKCLDNLCVNRQRDQVKRYYSVVYPGWPI
YPRNRAAARGGPEQKLISEEDLNSAVDHHHHHH
SEQ ID No 2: Core GLIPR1 Protein with RNR
METDTLLLWVLLLWVPGSTGANILPDIENEDFIKDCVRIHNKFRSEVK
PTASDMLYMTWD PALAQIAKAWASNCQFSHNTRLKPPHKLHPNFTSL
GENIWTGSVPIFSVSSAITNWYDEIQDYDFKTRICKKVCGHYTQVVWA
DSYKVGCAVQFCPKVSGFDALSNGAHFICNYGPGGNYPTWPYKRGATC
SACPNNDKCLDNLCVNRQRDQVKRYYSVVYPGWPIYPRNR
SEQ ID No 3: Core GLIPR1 Protein without RNR
METDTLLLWVLLLWVPGSTGANILPDIENEDFIKDCVRIHNKFRSEVK
PTASDMLYMTWDPALAQIAKAWASNCQFSHNTRLKPPHKLHPNFTSLG
ENIWTGSVPIFSVSSAITNWYDEIQDYDFKTRICKKVCGHYTQVVWAD
SYKVGCAVQFCPKVSGFDALSNGAHFICNYGPGGNYPTWPYKRGATCS
ACPNNDKCLDNLCVNRQRDQVKRYYSVVYPGWPIYP
SEQ ID No 4: Core GLIPR1 Protein with DAAP and RNR
METDTLLLWVLLLWVPGSTGDAAPANILPDIENEDFIKDCVRIHNKFR
SEVKPTASDMLYMTWDPALAQIAKAWASNCQFSHNTRLKPPHKLHPNF
TSLGENIWTGSVPIFSVSSAITNWYDEIQDYDFKTRICKKVCGHYTQV
VWADSYKVGCAVQFCPKVSGFDALSNGAHFICNYGPGGNYPTWPYKRG
ATCSACPNNDKCLDNLCVNRQRDQVKRYYSVVYPGWPIYPRNR
SEQ ID No 5: Core GLIPR1 Protein with DAAP and
without RNR
METDTLLLWVLLLWVPGSTGDAAPANILPDIENEDFIKDCVRIHNKFR
SEVKPTASDMLYMTWDPALAQIAKAWASNCQFSHNTRLKPPHKLHPNF
TSLGENIWTGSVPIFSVSSAITNWYDEIQDYDFKTRICKKVCGHYTQV
VWADSYKVGCAVQFCPKVSGFDALSNGAHFICNYGPGGNYPTWPYKRG
ATCSACPNNDKCLDNLCVNRQRDQVKRYYSVVYPGWPIYP
SEQ ID No 6: Fc Fusion protein with RNR
METDTLLLWVLLLWVPGSTGANILPDIENEDFIKDCVRIHNKFRSEVK
PTASDMLYMTWDPALAQIAKAWASNCQFSHNTRLKPPHKLHPNFTSLG
ENIWTGSVPIFSVSSAITNWYDEIQDYDFKTRICKKVCGHYTQVVWAD
SYKVGCAVQFCPKVSGFDALSNGAHFICNYGPGGNYPTWPYKRGATCS
ACPNNDKCLDNLCVNRQRDQVKRYYSVVYPGWPIYPRNRDKTHTCPPC
PAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFY
PSDIAVEWESNGQPENNYKTTPPVLDSDGSFFLYSKLTVDKSRWQQGN
VFSCSVMHEALHNHYTQKSLSLSPGK
SEQ ID No 7: Fe Fusion protein without RNR
METDTLLLWVLLLWVPGSTGANILPDIENEDFIKDCVRIHNKFRSEVK
PTASDMLYMTWDPALAQIAKAWASNCQFSHNTRLKPPHKLHPNFTSLG
ENIWTGSVPIFSVSSAITNWYDEIQDYDFKTRICKKVCGHYTQVVWAD
SYKVGCAVQFCPKVSGFDALSNGAHFICNYGPGGNYPTWPYKRGATCS
ACPNNDKCLDNLCVNRQRDQVKRYYSVVYPGWPIYPDKTHTCPPCPAP
ELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFS
CSVMHEALHNHYTQKSLSLSPGK
SEQ ID No 8: Fc Fusion protein with DAAP and RNR
METDTLLLWVLLLWVPGSTGDAAPANILPDIENEDFIKDCVRIHNKFR
SEVKPTASDMLYMTWDPALAQIAKAWASNCQFSHNTRLKPPHKLHPNF
TSLGENIWTGSVPIFSVSSAITNWYDEIQDYDFKTRICKKVCGHYTQV
VWADSYKVGCAVQFCPKVSGFDALSNGAHFICNYGPGGNYPTWPYKRG
ATCSACPNNDKCLDNLCVNRQRDQVKRYYSVVYPGWPIYPRNRDKTHT
CPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLV
KGFYPSDIAVEWESNGQPENNYKTTPPVLDSDGSFFLYSKLTVDKSRW
QQGNVFSCSVMHEALHNHYTQKSLSLSPGK
SEQ ID No 9: Fe Fusion protein with DAAP and
without RNR
METDTLLLWVLLLWVPGSTGDAAPANILPDIENEDFIKDCVRIHNKFR
SEVKPTASDMLYMTWDPALAQIAKAWASNCQFSHNTRLKPPHKLHPNF
TSLGENIWTGSVPIFSVSSAITNWYDEIQDYDFKTRICKKVCGHYTQV
VWADSYKVGCAVQFCPKVSGFDALSNGAHFICNYGPGGNYPTWPYKRG
ATCSACPNNDKCLDNLCVNRQRDQVKRYYSVVYPGWPIYPDKTHTCPP
CPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGF
YPSDIAVEWESNGQPENNYKTTPPVLDSDGSFFLYSKLTVDKSRWQQG
NVFSCSVMHEALHNHYTQKSLSLSPGK
SEQ ID No 10: Fc-Glipr1: +DAAP/+RNR
DAAPANILPDIENEDFIKDCVRIHNKFRSEVKPTASDMLYMTWDPALA
QIAKAWASNCQFSHNTRLKPPHKLHPNFTSLGENIWTGSVPIFSVSSA
ITNWYDEIQDYDFKTRICKKVCGHYTQVVWADSYKVGCAVQFCPKVSG
FDALSNGAHFICNYGPGGNYPTWPYKRGATCSACPNNDKCLDNLCVNR
QRDQVKRYYSVVYPGWPIYPRNRDKTHTCPPCPAPELLGGPSVFLFPP
KPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKG
QPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYT
QKSLSLSPGK
SEQ ID No 11: Fc-Gliprl: +DAAP/−RNR
DAAPANILPDIENEDFIKDCVRIHNKFRSEVKPTASDMLYMTWDPALA
QIAKAWASNCQFSHNTRLKPPHKLHPNFTSLGENIWTGSVPIFSVSSA
ITNWYDEIQDYDFKTRICKKVCGHYTQVVWADSYKVGCAVQFCPKVSG
FDALSNGAHFICNYGPGGNYPTWPYK RGATCSACPNNDKCLDNLCVNR
QRDQVKRYYSVVYPGWPIYP---DKTHTCPPCPAPELLGGPSVFLFPP
KPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKG
QPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYT
QKSLSLSPGK
SEQ ID No 12: Fc-Glipr1: −DAAP/+RNR
ANILPDIENEDFIKDCVRIHNKFRSEVKPTASDMLYMTWDPALAQIAK
AWASNCQFSHNTRLKPPHKLHPNFTSLGENIWTGSVPIFSVSSAITNW
YDEIQDYDFKTRICKKVCGHYTQVVWADSYKVGCAVQFCPKVSGFDAL
SNGAHFICNYGPGGNYPTWPYKRGATCSACPNNDKCLDNLCVNRQRDQ
VKRYYSVVYPGWPIYPRNRDKTHTCPPCPAPELLGGPSVFLFPPKPKD
TLMISRTPEVTCVVVDVSHEDPEVKFNWYVDGVEVHNAKTKPREEQYN
STYRVVSVLTVLHQDWLNGKEYKCKVSNKALPAPIEKTISKAKGQPRE
PQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSL
SLSPGK
SEQ ID No 13: Fc-Glipr1: −DAAP/−RNR
ANILPDIENEDFIKDCVRIHNKFRSEVKPTASDMLYMTWDPALAQIAK
AWASNCQFSHNTRLKPPHKLHPNFTSLGENIWTGSVPIFSVSSAITNW
YDEIQDYDFKTRICKKVCGHYTQVVWADSYKVGCAVQFCPKVSGFDAL
SNGAHFICNYGPGGNYPTWPYK RGATCSACPNNDKCLDNLCVNRQRD
QVKRYYSVVYPGWPIYP---DKTHTCPPCPAPELLGGPSVFLFPPKPK
DTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKS
LSLSPGK
SEQ ID No 14: Fc-Glipr1: Glycine linker only
DAAPANILPDIENEDFIKDCVRIHNKFRSEVKPTASDMLYMTWDPALA
QIAKAWASNCQFSHNTRLKPPHKLHPNFTSLGENIWTGSVPIFSVSSA
ITNWYDEIQDYDFKTRICKKVCGHYTQVVWADSYKVGCAVQFCPKVSG
FDALSNGAHFICNYGPGGNYPTWPYKRGATCSACPNNDKCLDNLCVNR
QRDQVGGGGS------------DKTHTCPPCPAPELLGGPSVFLFPPK
PKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQ
KSLSLSPGK
SEQ ID No 15: Fc-Glipr1: linker
DAAPANILPDIENEDFIKDCVRIHNKFRSEVKPTASDMLYMTWDPALA
QIAKAWASNCQFSHNTRLKPPHKLHPNFTSLGENIWTGSVPIFSVSSA
ITNWYDEIQDYDFKTRICKKVCGHYTQVVWADSYKVGCAVQFCPKVSG
FDALSNGAHFICNYGPGGNYPTWPYKRGATCSACPNNDKCLDNLCVNR
QRDQV------------------DKTHTCPPCPAPELLGGPSVFLFPP
KPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKG
QPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYT
QKSLSLSPGK
SEQ ID No 16: his-tag Glipr1
DAAQPARRARRTKLANILPDIENEDFIKDCVRIHNKFRSEVKPTASDM
LYMTWDPALAQIAKAWASNCQFSHNTRLKPPHKLHPNFTSLGENIWTG
SVPIFSVSSAITNWYDEIQDYDFKTRICKKVCGHYTQVVWADSYKVGC
AVQFCPKVSGFDALSNGAHFICNYGPGGNYPTWPYKRGATCSACPNND
KCLDNLCVNRQRDQVKRYYSVVYPGWPIYPRNRAAARGGPEQKLISEE
DLNSAVDHHHHHH
SEQ ID No 17: native Glipr1 (transmembrane)
ANILPDIENEDFIKDCVRIHNKFRSEVKPTASDMLYMTWDPALAQIAK
AWASNCQFSHNTRLKPPHKLHPNFTSLGENIWTGSVPIFSVSSAITNW
YDEIQDYDFKTRICKKVCGHYTQVVWADSYKVGCAVQFCPKVSGFDAL
SNGAHFICNYGPGGNYPTWPYKRGATCSACPNNDKCLDNLCVNRQRDQ
VKRYYSVVYPGWPIYPRNRYTSLFLIVNSVILILSVIITILVQHKYPN
LVLLD
SEQ ID No 18: METDTLLLWVLLLWVPGSTG (IgK leader)
SEQ ID No 19: DAAQPARRARRTKL (linker)
SEQ ID No 20: EQKLISEEDL (myc tag)
SEQ ID No 21: (GLIPR1 CH2 domain)
ELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKAL
PAPIEKTISKAK
SEQ ID No 22: (GLIPR1 CH3 domain)
GQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHY
TQKSLSLSPGK
SEQ ID No 23: Antibody F(ab)2 pepsin digestion
product
DAAPANILPDIENEDFIKDCVRIHNKFRSEVKPTASDMLYMTWDPALA
QIAKAWASNCQFSHNTRLKPPHKLHPNFTSLGENIWTGSVPIFSVSSA
ITNWYDEIQDYDFKTRICKKVCGHYTQVVWADSYKVGCAVQFCPKVSG
FDALSNGAHFICNYGPGGNYPTWPYKRGATCSACPNNDKCLDNLCVNR
QRDQVKRYYSVVYPGWPIYPRNRDKTHTCPPCPAPE

1. A recombinant peptide comprising a first amino acid sequence of a GLIPR protein coupled to a second amino acid sequence that is not obtained or derived from the GLIPR protein.

2. The peptide of claim 1, wherein the first amino acid sequence comprises a conserved region of the GLIPR protein.

3. The peptide of claim 1, wherein the first amino acid sequence comprises a domain of the GLIPR protein.

4. The peptide of claim 3, wherein the domain comprises a transmembrane domain, a secretory domain, or an extracellular domain, or a functional subdomain thereof.

5. The peptide of claim 1, wherein the first amino acid sequence comprises any one of SEQ ID Nos. 2-5 or a sequence that is at least 90% identical to any one of SEQ ID NOs 2-5.

6. The peptide of claim 1, which comprises any one of SEQ ID NOs 1 and 6-17 or a sequence that is at least 90% identical to any one of SEQ ID NOs 1 and 6-17.

7. The peptide of claim 1, wherein the GLIPR protein comprises human or murine GLIPR1 protein.

8. The peptide of claim 7, wherein the human GLIPR protein comprises GLIPR1, GLIPR1L1, GLIPR1L2, GLIPR 1 alpha, GLIPR1 beta, GLIPR2alpha, GLIPR2beta, GLIPR2gamma, GLIPR2delta, or GLIPR2episilon.

9. The peptide of claim 1, wherein the second amino acid sequence comprises a sequence obtained or derived from an antibody, an immunological protein, an immune-regulatory protein, a cytokine, or a toxin protein.

10. The peptide of claim 9, wherein the antibody is an IgA, an IgD, an IgE, an IgG, or an IgM.

11. The composition of claim 1, wherein the second amino acid sequence is obtained or derived from an Fc region of the antibody.

12. The composition of claim 1, which has a circulating half-life of at least ten times the circulating half-life of the GLIPR protein.

13. The composition of claim 1, which has a circulating half-life of at least one hundred time the circulating half-life of the GLIPR protein.

14. A recombinant nucleic acid that encodes the peptide of claim 1.

15. An expression vector comprising the nucleic acid of claim 14.

16. A method for treating a patient comprising:
providing a composition containing the peptide of claim 1; and
administering a therapeutic amount of the composition to the patient.

17. The method of claim 16, wherein the patient has a disorder associated with an uncontrolled growth of cells.

18. The method of claim 17, wherein the uncontrolled growth of cells comprises a malignancy.

19. The method of claim 18, wherein the malignancy comprises bone, bladder, kidney, liver, lung, or prostate cancer.

20. The method of claim 16, wherein the patient has a reduced expression of GLIPR1 protein in cells as compared to similar cells in which GLIPR1 protein expression is normally enhanced.

21. The method of claim 16, wherein administering comprises systemic administration to the bloodstream of the patient.

22. The method of claim 16, wherein the uncontrolled growth of cells comprises a tumor.

23. The method of claim 22, wherein the tumor comprises a bone, a bladder, a kidney, a liver, a lung, or a prostate tumor.

24. The method of claim 22, wherein administering comprises local administration to the tumor.

25. A peptide comprising a first amino acid sequence of a GLIPR core protein coupled to a second amino acid sequence comprising a Fc portion of an antibody.

26. The peptide of claim 25, which has a circulating half-life that is significantly greater than the circulating half-life of the GLIPR core protein.

27. The peptide of claim 25, which has a circulating half-life that is at least ten times greater than the circulating half-life of the GLIPR core protein.

28. The peptide of claim 25, which has a circulating half-life that is at least one hundred times greater than the circulating half-life of the GLIPR core protein.

29. The peptide of claim 25, wherein the first amino acid sequence comprises any one of SEQ ID NOs. 2-5, or a sequence that is at least 90% identical to any one of SEQ ID NOs 2-5.

30. The peptide of claim 25, which comprises the sequence of any one of SEQ ID NO. 1 and 6-15, or a sequence that is at least 90% identical to any one of SEQ ID NOs 1 and 6-15.

31. A method of expressing a peptide comprising:
providing a recombinant vector; and
incubating the recombinant vector in a protein expression system, and
expression the peptide, wherein the peptide contains a first amino acid sequence of a GLIPR protein coupled to a second amino acid sequence that is not obtained or derived from the GLIPR protein with DAAP coupled to the N-terminus.


SEQUENCE LISTING (24 sequences)

SEQ ID NO: 1 - protein, 273 aa, Artificial Sequence
  Description of Artificial Sequence Synthetic polypeptide
METDTLLLWVLLLWVPGSTGDAAQPARRARRTKLANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRAAARGGPEQKLISEEDLNSAVDHHHHHH

SEQ ID NO: 2 - protein, 231 aa, Artificial Sequence
  Description of Artificial Sequence Synthetic polypeptide
METDTLLLWVLLLWVPGSTGANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNR

SEQ ID NO: 3 - protein, 228 aa, Artificial Sequence
  Description of Artificial Sequence Synthetic polypeptide
METDTLLLWVLLLWVPGSTGANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYP

SEQ ID NO: 4 - protein, 235 aa, Artificial Sequence
  Description of Artificial Sequence Synthetic polypeptide
METDTLLLWVLLLWVPGSTGDAAPANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNR

SEQ ID NO: 5 - protein, 232 aa, Artificial Sequence
  Description of Artificial Sequence Synthetic polypeptide
METDTLLLWVLLLWVPGSTGDAAPANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYP

SEQ ID NO: 6 - protein, 458 aa, Artificial Sequence
  Description of Artificial Sequence Synthetic polypeptide
METDTLLLWVLLLWVPGSTGANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 455 aa, Artificial Sequence
  Description of Artificial Sequence Synthetic polypeptide
METDTLLLWVLLLWVPGSTGANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 462 aa, Artificial Sequence
  Description of Artificial Sequence Synthetic polypeptide
METDTLLLWVLLLWVPGSTGDAAPANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 459 aa, Artificial Sequence
  Description of Artificial Sequence Synthetic polypeptide
METDTLLLWVLLLWVPGSTGDAAPANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 442 aa, Artificial Sequence
  Description of Artificial Sequence Synthetic polypeptide
DAAPANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 439 aa, Artificial Sequence
  Description of Artificial Sequence Synthetic polypeptide
DAAPANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 438 aa, Artificial Sequence
  Description of Artificial Sequence Synthetic polypeptide
ANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 435 aa, Artificial Sequence
  Description of Artificial Sequence Synthetic polypeptide
ANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 429 aa, Artificial Sequence
  Description of Artificial Sequence Synthetic polypeptide
DAAPANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 424 aa, Artificial Sequence
  Description of Artificial Sequence Synthetic polypeptide
DAAPANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 253 aa, Artificial Sequence
  Description of Artificial Sequence Synthetic polypeptide
DAAQPARRARRTKLANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRAAARGGPEQKLISEEDLNSAVDHHHHHH

SEQ ID NO: 17 - protein, 245 aa, Artificial Sequence
  Description of Artificial Sequence Synthetic polypeptide
ANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRYTSLFLIVNSVILILSVIITILVQHKYPNLVLLD

SEQ ID NO: 18 - protein, 20 aa, Artificial Sequence
  Description of Artificial Sequence Synthetic peptide
METDTLLLWVLLLWVPGSTG

SEQ ID NO: 19 - protein, 14 aa, Artificial Sequence
  Description of Artificial Sequence Synthetic peptide
DAAQPARRARRTKL

SEQ ID NO: 20 - protein, 10 aa, Artificial Sequence
  Description of Artificial Sequence Synthetic peptide
EQKLISEEDL

SEQ ID NO: 21 - protein, 108 aa, Artificial Sequence
  Description of Artificial Sequence Synthetic polypeptide
ELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 22 - protein, 107 aa, Artificial Sequence
  Description of Artificial Sequence Synthetic polypeptide
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 228 aa, Artificial Sequence
  Description of Artificial Sequence Synthetic polypeptide
DAAPANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRDKTHTCPPCPAPE

SEQ ID NO: 24 - protein, 4 aa, Artificial Sequence
  Description of Artificial Sequence Synthetic peptide
DAAP